(19) United States
(12) Patent Application Publication
(10) Pub. No.: US 2025/0082669 A1
(43) Pub. Date: Mar. 13, 2025

(54) POVIDONE IODINE SOLUTION AND GEL, PREPARATION METHOD AND APPLICATION THEREOF

(71) Applicant: Dukal, LLC, Ronkonkoma, NY (US)

(72) Inventor: La YANG, Dunedin, FL (US)

(21) Appl. No.: 18/725,506

(22) PCT Filed: Dec. 28, 2022
(86) PCT No.: PCT/US2022/054161

§ 371 Date: Jun. 28, 2024

(30) Foreign Application Priority Data
Dec. 29, 2021 (CN) 202111643764.9
Dec. 29, 2021 (CN) 202111647333.X

(51) Int. Cl. A61K 31/79; A61K 9/06; A61P 31/02

(52) U.S. Cl. CPC A61K 31/79; A61K 9/06; A61P 31/02

(57) ABSTRACT

The present invention belongs to the field of antibacterial and disinfectant preparations, and relates to a povidone iodine solution and gel with enhanced stability, and a preparation method and application thereof. The povidone iodine solution includes povidone iodine, a stabilizer bag and a pH regulator bag. Specifically, the povidone iodine solution includes the following components based on parts by weight: 5-15 parts by weight of povidone iodine, 0.4-1.8 parts by weight of stabilizer bag, 0.6-1.0 part by weight of pH regulator bag and the balance purified water. Specifically, the pH value of the povidone iodine solution is 1.5-6.5. The provide iodine gel includes povidone iodine, a gel matrix, a stabilizer pack, and a pH regulator pack.

### CROSS-REFERENCE TO RELATED APPLICATIONS

The present application claims benefit of and priority to Chinese Patent Application No. 20211164733.X filed Dec. 29, 2021 and Chinese Patent Application No. 202111643764.9 filed Dec. 29, 2021, the entire content of each of which is hereby incorporated by reference herein.

### BACKGROUND

### Field of the Disclosure

The present invention belongs to the field of antibacterial and disinfectant preparations, and relates to a povidone iodine solution, a preparation method and application thereof. The present invention also relates to a water-soluble povidone iodine gel and a preparation method and application thereof.

### Related Art

Povidone iodine, as a broad-spectrum and efficient disinfectant preservative, is a loose compound formed by polyvinylpyrrolidone (PVP) and iodine in a complexing way. PVP not only serves as a carrier, but also has a dissolution assisting function. When in contact with a wound surface or an affected part, the povidone iodine gradually releases free iodine, thereby playing a slow release role and keeping a bactericidal capacity for a long time. Its bactericidal mechanism is that the free iodine oxidizes active groups of microbial protein, and can be combined with amino groups of the protein to denature the protein. The povidone iodine, as a broad-spectrum antibacterial and disinfectant preparation, has an eliminating effect on various bacteria, spores, viruses, fungi and the like, has small irritation to skin, low toxicity and lasting effect, is safe and convenient to use, basically has no irritation to tissues, and is commonly used for disinfection of skin and mucosa. A povidone iodine aqueous solution has no use taboos similar to iodine tincture, and it has light color, is easy to elute, has small irritation to mucosa, does not need ethanol deiodination, has no corrosion effect, and has low toxicity. The “Chinese Pharmacopoeia” 2020 Edition (Volume II) records the standards of povidone iodine solution, agent and other dosage forms, and requires that the effective iodine is 8.5% to 12.0% of the labeled amount.

However, in the storage process of the povidone iodine, iodine will be continuously dissociated, the iodine reacts with water to generate HIO⁻ and I⁻, and the HIO can further generate HIO₃⁻, so that the effective iodine content is continuously reduced, and as a result, the product does not meet the standard requirements.

An existing patent document CN104906127A discloses a povidone iodine compound preparation with enhanced effective iodine content stability and a preparation method therefor. According to the method, the effective iodine content in the preparation can be kept relatively stable by adding sodium hydroxide, but this method only considers the influence on the stability of a low-concentration povidone iodine (2-5%) preparation, and does not research the stability of an existing povidone iodine solution for human skin surface disinfectant, and the concentration of these products is mainly 5-15%. In this the method, since abundant ethanol is used as a solvent, the production cost is high, and moreover, the ethanol will stimulate the wound in the use process, thereby causing poor use compliance of a patient. Meanwhile, the method does not provide the product bactericidal effect data, and so it cannot confirm whether the povidone solution has favorable bactericidal capacity.

In addition, an existing patent document CN112315974A discloses a povidone iodine solution with enhanced stability and a preparation method therefor. According to the method, sodium dihydrogen phosphate and sodium phosphate dibasic dodecahydrate are added as pH value regulation buffer pairs, potassium iodate and lauryl sodium sulfate are used as pH stabilizers, and sodium hydroxide is used as a pH regulator. However, this method only considers the influence on the stability of a low-concentration povidone iodine preparation (0.5-2%), and does not consider the stability of the existing povidone iodine solution for human skin surface disinfection in the market, and the concentration of these products in practical application is mainly 5-15%. Besides, the method only investigates the stability data for 12 m, and cannot judge or verify the product stability for longer time. Meanwhile, too many pH regulators are used in the document, so the preparation process is tedious and complex.

In view of the defects and deficiencies of existing povidone iodine related products, the inventor of the present invention expects to improve a disinfectant preparation with povidone iodine as a main component and simplify a preparation method. Specifically, the present invention provides a simple preparation method and a novel povidone iodine solution prepared therefrom, and the novel povidone iodine solution has good adhesion, enhanced stability and bactericidal capacity.

Further, existing povidone iodine solutions, due to their fluidity make it difficult for such solutions to fully attach to the skin, as drips are likely such that the portion of the product that actually provides disinfection is greatly reduced. In addition, the fluidity of the solution also makes it more inconvenient to use. In the prior art, according to a Chinese patent application with a publication number of CN112451474A, a povidone iodine gel and a preparation method are disclosed. According to the method, a povidone gel is prepared by using chitosan as a gel medium. In the method, a large amount of ethanol is used as a solvent, so that not only is the production cost high, but also a wound is stimulated by the ethanol during use, resulting in poor use compliance of a patient. In addition, according to the method, specific results of the stability and bactericidal effect of a product are not provided, and whether the povidone gel has good stability and bactericidal ability cannot be confirmed.

Accordingly it would be desirable to provide povidone iodine related products for sterilization and disinfection that avoid these and other problems.

### SUMMARY

It is an object of the present disclosure to provide a povidone iodine solution, a preparation method and application thereof in preparation of disinfectant preparations.

In embodiments, a povidone iodine solution includes povidone iodine, a stabilizer bag and a pH regulator bag.

In embodiments, the povidone iodine solution, based on 100 parts by weight, includes:
5-15 parts by weight of povidone iodine;
0.4-1.8 parts by weight of the stabilizer bag;
0.6-1.0 part by weight of the pH regulator bag; and
the balance water; where the pH value of the povidone iodine solution is 1.5-6.5.

In embodiments, the stabilizer bag at least includes a first stabilizer for protecting a polymer and a second stabilizer for reducing iodine decomposition, preferably, a nonionic surfactant is selected as the first stabilizer, and potassium iodide or sodium iodide is selected as the second stabilizer.

In embodiments, the stabilizer bag includes 0.1-1.0 part by weight of the first stabilizer and 0.3-0.8 part by weight of the second stabilizer.

In embodiments, the first stabilizer is any one selected from the nonionic surfactants. In embodiments, the first stabilizer may be nonylphenol polyoxyethylene ether, more preferably, nonylphenol polyoxyethylene ether-9 or nonylphenol polyoxyethylene ether-10.

Further, in embodiments, the pH regulator bag includes acid and alkali, the acid includes any one of citric acid and oxalic acid, and the alkali includes any one of sodium hydroxide, potassium hydroxide and sodium carbonate; and preferably, the pH regulator bag includes 0.3-0.5 part by weight of citric acid and 0.3-0.5 part by weight of sodium hydroxide.

In embodiments, effective iodine content of the povidone iodine solution decreases by less than 10% after 24 months storage at 25±2° C. and 60%±5% relative humidity.

In embodiments, effective iodine content of the povidone iodine solution decreases by less than 20% after 36 months storage at 25±2° C. and 60%±5% relative humidity.

A method for preparing a povidone iodine solution, in accordance with an embodiment of the present disclosure, includes:
(1) providing the following raw material components by weight as follows:
   5-15 parts by weight of povidone iodine;
   0.4-1.8 parts by weight of the stabilizer bag; and
   0.6-1.0 part by weight of the pH regulator bag;
(2) adding 5-15 parts by weight of povidone iodine into purified water, and performing stirring till complete dissolution, thereby obtaining a first intermediate solution; and
(3) adding the stabilizer bag and the pH regulator bag into the first intermediate solution respectively, and performing stirring, thereby obtaining the povidone iodine solution.

In embodiments, the method includes weighing the component raw materials.

In embodiments, the stabilizer bag at least includes the first stabilizer for protecting the polymer and the second stabilizer for reducing iodine decomposition, wherein nonylphenol polyether is selected as the first stabilizer, and potassium iodide is selected as the second stabilizer.

In embodiments, the pH regulator bag includes acid and alkali, wherein the acid includes any one of citric acid and oxalic acid, and the alkali includes any one of sodium hydroxide, potassium hydroxide and sodium carbonate; and preferably, the pH regulator bag includes 0.3-0.5 part by weight of citric acid and 0.3-0.5 part by weight of sodium hydroxide.

In embodiments, the present invention also provides a disinfection product taking a non-woven fabric or a medical sponge as a carrier to carry the povidone iodine solution.

In embodiments, a method of killing 80% or more of a bacteria in or on a substrate comprising contacting the substrate with an amount of the povidone iodine solution.

In embodiments, 90% or more of the bacteria are killed.

In embodiments, 88% or more of the bacterial are killed.

In embodiments, the bacteria are one or more of:
A. baumanii
B. fragilis
C. albicans
C. tropicalis
E. aerogenes
E. coli
E. coli
H. influenzae
K. pneumoniae
P. mirabilis
P. aeruginosa
P. aeruginosa
S. marcesens
S. aureus
S. aureus
S. epidermidis
S. haemolyticus
S. hominis
S. saprophyticus
S. pneumoniae, and
S. pyogenes.

In embodiments, a ready-to-use disinfection cotton swab uses a cotton swab as a carrier to carry the povidone iodine solution. In embodiments, a ready-to-use disinfectant tablet uses a non-woven fabric as a carrier to carry the povidone iodine solutions. In embodiments, the present invention also provides a ready-to-use disinfection sponge, which uses medical sponge as a carrier to carry the povidone iodine solution.

In the above-mentioned technical solution of the present invention, the purified water is used as the solvent, which does not cause stimulation to the wounds; citric acid and sodium hydroxide are used as the pH regulators; the surfactant that is nonylphenol polyether is used as the stabilizer for protecting the polymer; and potassium iodide is used as the stabilizer for reducing effective iodine decomposition. The povidone iodine solution provided by the present invention has good adhesiveness, enhanced stability and bactericidal capacity. Meanwhile, the povidone iodine solution provided by the present invention can be directly used as the disinfectant preparation, and can also be added to different carriers as required to prepare different products. The povidone iodine solution has excellent stability, and is convenient for storage, transportation and use.

In embodiments, the present disclosure provides a novel friendly broad-spectrum disinfection gel and a preparation method and application thereof, and more specifically relates to a water-soluble povidone iodine gel and a preparation method and application thereof in preparation of a disinfection preparation.

In embodiments, the present disclosure provides a water-soluble povidone iodine gel. The povidone iodine gel includes povidone iodine, a gel matrix, a stabilizer pack, and a pH regulator pack.

In embodiments, in parts by weight, the povidone iodine gel includes:
5-15 parts by weight of the povidone iodine,
0.3-1.5 parts by weight of the gel matrix,
0.4-1.8 parts by weight of the stabilizer pack,
0.6-1.0 part by weight of the pH regulator pack, and
the balance of purified water;
wherein the povidone iodine gel has a pH value of 1.5-6.5 and a viscosity of 400-5,000 mPa·s.

In embodiments, the povidone iodine raw material has an available iodine content of 9.0-12.0%.

In embodiments, the gel matrix is selected from any one of nonionic soluble cellulose ethers or carbomer.

In embodiments, the gel matrix is hydroxyethyl cellulose, and the hydroxyethyl cellulose has a viscosity of 1,500-6,500 mPa·s.

In embodiments, the stabilizer pack at least includes a first stabilizer for protecting a polymer and a second stabilizer for reducing decomposition of iodine. Preferably, a nonionic surfactant is selected as the first stabilizer, and potassium iodide or sodium iodide is selected as the second stabilizer.

In embodiments, the stabilizer pack includes 0.1-1.0 part by weight of the first stabilizer and 0.3-0.8 part by weight of the second stabilizer.

In embodiments, the first stabilizer is selected from any one of nonionic surfactants.

In embodiments, the first stabilizer is nonylphenol polyoxyethylene ether, and more preferably nonylphenol polyoxyethylene ether-9 or nonylphenol polyoxyethylene ether-10.

In embodiments, the pH regulator pack includes an acid and an alkali. The acid includes any one of citric acid and oxalic acid. The alkali includes any one of sodium hydroxide, potassium hydroxide, and sodium carbonate. Preferably, the pH regulator pack includes 0.3-0.5 part by weight of the citric acid and 0.3-0.5 part by weight of the sodium hydroxide.

In embodiments, the povidone iodine gel further includes glycerol, preferably 0.05-1.0 part by weight of glycerol.

In embodiments, the effective iodine content of the povidone iodine gel decreases by less than 15% after 24 months storage at 25±2° C. and 60%±5% relative humidity.

In embodiments, the effective iodine content of the povidone iodine gel decreases by less than 20% after 36 months storage at 25±2° C. and 60%±5% relative humidity.

In an embodiment, the present disclosure provides a method for preparing a water-soluble povidone iodine gel including the following steps:
(1) providing the following component raw materials according to the following parts by weight: 5-15 parts by weight of povidone iodine, 0.3-1.5 parts by weight of a gel matrix, 0.4-1.8 parts by weight of a stabilizer pack, 0.6-1.0 part by weight of a pH regulator pack, and the balance of water, where the stabilizer pack includes a first stabilizer and a second stabilizer, and the pH regulator pack includes an acid and an alkali;
(2) separately conduction dissolution in a small amount of purified water to obtain a first stabilizer solution, a second stabilizer solution, an acid solution, and an alkali solution;
(3) adding purified water to a liquid preparation tank; slowly adding the gel matrix to the purified water under the condition of homogeneous stirring; after the addition is completed, conducting vacuumizing to −0.05 MPa to −0.5 MPa; and then conducting stirring to restore to a normal pressure state so as to obtain a first intermediate solution;
(4) adding 5-15 parts by weight of the povidone iodine to the first intermediate solution, and conducting standing; adding the balance of the water, and conducting vacuumizing to 0.05-0.5 MPa; and then conducting homogeneous stirring for mixing and stirring until complete dissolution to restore to a normal pressure state; and
(5) adding the first stabilizer solution and the second stabilizer solution to the liquid preparation tank containing the first intermediate solution, conducting mixing and stirring until complete dissolution, and adding the acid solution and the alkali solution to adjust the pH to 1.5-6.5 so as to obtain the water-soluble povidone iodine gel.

In embodiments, the method may include weighing the component raw materials.

In embodiments, the pH regulator pack includes an acid and an alkali. The acid includes any one of citric acid and oxalic acid. The alkali includes any one of sodium hydroxide, potassium hydroxide, and sodium carbonate. Preferably, the pH regulator pack includes 0.3-0.5 part by weight of the citric acid and 0.3-0.5 part by weight of the sodium hydroxide.

In embodiments, a disinfection preparation containing the povidone iodine gel, where the povidone iodine gel has a content of 5-15 weight %.

In embodiments, the disinfection preparation is concentrated and/or ready-to-use.

In embodiments, the present disclosure provides a ready-to-use disinfection cotton swab used as a carrier to load a disinfection preparation containing the povidone iodine gel.

In embodiments, the present disclosure provides a ready-to-use disinfection paste, wherein a non-woven fabric is used as a carrier to load a disinfection preparation containing the povidone iodine gel.

In embodiments, a method of killing 80% or more of a bacteria in or on a substrate comprising contacting the substrate with an amount of the povidone iodine gel in an amount sufficient to kill the bacteria.

In embodiments, 90% or more of the bacteria are killed.

In embodiments, 99% or more of the bacteria are killed.

In embodiments, the bacteria are one or more of the following:
Acinetobacter baumannii
Bacteroides fragilis
Candida albicans
Candida tropicalis
Enterococcus faecium
Enterobacter cloacae
Escherichia coli
Escherichia coli
Haemophilus influenzae
Klebsiella pneumoniae
Proteus mirabilis
Pseudomonas aeruginosa
Pseudomonas aeruginosa
Serratia marcesens
Staphylococcus aureus
Staphylococcus aureus
Staphylococcus epidermidis
Staphylococcus haemolyticus
Staphylococcus hominis
Staphylococcus saprophyticus, and
Streptococcus pneumoniae.

### DETAILED DESCRIPTION OF THE EXEMPLARY EMBODIMENTS

The technical solution of the present invention will be further described in conjunction with specific implementations, but this does not limit the present invention. On the premise of not deviating from the technical concept and technical solution of the present invention, any modification, adjustment or modification, or equivalent replacement method that can be realized for ordinary technicians in the art to which the present invention belongs will fall into the scope of protection required by the present invention.

Unless otherwise specified, experimental methods used in the following examples are conventional methods. Unless otherwise specified, materials, reagents and the like used in the following examples may be commercially available.

In the above technical schemes of the present invention, the purified water is used as a solvent, so that a wound is not stimulated. The hydroxyethyl cellulose is used as the gel matrix. The citric acid and the sodium hydroxide are used as a pH regulator. The surfactant, namely nonylphenol polyether, is used as a stabilizer for protecting a polymer. The potassium iodide is used as a stabilizer for reducing decomposition of available iodine. The povidone iodine gel provided by the present invention has good adhesion, stability, and bactericidal ability. Meanwhile, the povidone iodine gel of the present invention can be directly used as a gel disinfection preparation, and can also be added to different carriers as required to prepare other different disinfection products. The povidone iodine gel has excellent stability, and is convenient to store, transport and use.

### Beneficial Effects

The present invention discloses a povidone iodine gel and a preparation method thereof, a disinfection preparation containing the povidone iodine gel and application thereof. Based on examples and test results, the povidone iodine gel specifically has excellent adhesion, stability, and bactericidal ability.

The present invention also relates to the povidone iodine solution, the preparation method and the application thereof. Based on the embodiment and test result, the povidone iodine solution and other disinfection products containing the povidone iodine solution have excellent adhesiveness, enhanced stability and bactericidal capacity.

### Povidone Iodine Solution

In the embodiments of the present invention, in a first aspect, the present invention provides a povidone iodine solution, which at least includes povidone iodine, a stabilizer bag and a pH regulator bag.

Further, based on 100 parts by weight, the povidone iodine solution includes: 5-15 parts by weight of povidone iodine, 0.4-1.8 parts by weight of the stabilizer bag, 0.6-1.0 part by weight of the pH regulator bag, and the balance purified water, wherein the pH value of the povidone iodine solution is 1.5-6.5. In embodiments, the povidone iodine solution may include: 5-10 parts by weight of povidone iodine, 0.4-1.0 part by weight of the stabilizer bag, 0.6-0.8 part by weight of the pH regulator bag, and the balance purified water, wherein the pH value of the povidone iodine solution is 1.5-4.0. In embodiments, the povidone iodine solution may include 10-15 parts by weight of povidone iodine, 1.0-1.8 parts by weight of the stabilizer bag, 0.8-1.0 part by weight of the pH regulator bag, and the balance purified water, wherein the pH value of the povidone iodine solution is 4.0-6.5.

Further, the stabilizer bag at least includes a first stabilizer for protecting a polymer and a second stabilizer for reducing iodine decomposition, preferably, nonylphenol polyether is selected as the first stabilizer, and potassium iodide is selected as the second stabilizer. Preferably, the stabilizer bag includes 0.1-1.0 part by weight of the first stabilizer and 0.3-0.8 parts by weight of the second stabilizer. In embodiments, the stabilizer bag includes 0.1-0.5 part by weight of the first stabilizer and 0.3-0.5 part by weight of the second stabilizer. In embodiments, the stabilizer bag includes 0.5-1.0 part by weight of the first stabilizer and 0.5-0.8 parts by weight of the second stabilizer.

Further, the first stabilizer is any one selected from the nonionic surfactants, preferably, nonylphenol polyoxyethylene ether, more preferably, nonylphenol polyoxyethylene ether-9 or nonylphenol polyoxyethylene ether-10.

Further, the pH regulator bag includes acid and alkali, wherein the acid is any one of citric acid and oxalic acid, and the alkali is any one of sodium hydroxide, potassium hydroxide and sodium carbonate.

Further, the povidone iodine solution also includes glycerin, for example, 0.05-1.0 part by weight of glycerin. In embodiments, the povidone iodine solution includes 0.05-0.5 part by weight of glycerin. In embodiments, the povidone iodine solution includes 0.5-1.0 part by weight of glycerin.

In a second aspect, the present invention also provides a method for preparing the povidone iodine solution. The method includes the following steps:
(1) providing the following raw materials by weight:
   5-15 parts by weight of povidone iodine;
   0.4-1.8 parts by weight of the stabilizer bag; and
   0.6-1.0 part by weight of the pH regulator bag;
(2) adding 5-15 parts by weight of povidone iodine into purified water, and performing stirring till complete dissolution, thereby obtaining a first intermediate solution; and
(3) adding the stabilizer bag and the pH regulator bag into the first intermediate solution respectively, and performing stirring, thereby obtaining the povidone iodine solution.

In embodiments, the providing step may include weighing the raw materials.

Further, the pH regulator bag includes acid and alkali, the acid includes any one of citric acid and oxalic acid, and the alkali includes any one of sodium hydroxide, potassium hydroxide and sodium carbonate; and preferably, the pH regulator bag includes 0.3-0.5 part by weight of citric acid and 0.3-0.5 part by weight of sodium hydroxide.

Further, the acid and alkali in the pH regulator bag are respectively configured into an acid solution and an alkali solution, and a conventional acid solution and alkali solution are selectively added dropwise to regulate the pH value of the povidone iodine solution to be 1.5-6.5.

In a third aspect, the present invention also provides application of any one of the povidone iodine solutions in preparation of disinfection products.

Specifically, the present invention provides a ready-to-use disinfection cotton swab, which takes a cotton swab as a carrier to carry any one of the povidone iodine solutions. The present invention provides a ready-to-use disinfectant tablet, which takes a non-woven fabric as a carrier to carry any one of the povidone iodine solutions. The present invention also provides a ready-to-use disinfection sponge, which takes medical sponge as a carrier to carry any one of the povidone iodine solutions.

The povidone iodine solution and other products provided by the present invention are prepared and their performances are detected according to the following specific embodiments and experimental examples.

### Embodiment 1

A povidone iodine solution was prepared by the following steps:
(1) providing the following each raw material component by weight:
   7.5 kg of povidone iodine, 0.5 kg of potassium iodide, 0.5 kg of nonylphenol polyether-10, 0.4 kg of citric acid, 0.3 kg of sodium hydroxide, and 90.8 kg of purified water for standby use;
(2) adding povidone iodine into purified water, and performing stirring till complete dissolution, thereby obtaining a first intermediate solution; and
(3) adding a first stabilizer, a second stabilizer, citric acid and sodium hydroxide into the first intermediate solution respectively, and performing stirring, thereby obtaining the povidone iodine solution.

According to the steps, the prepared povidone iodine solution includes the following components by weight:
7.5 parts by weight of povidone iodine, 0.5 part by weight of potassium iodide, 0.5 part by weight of nonylphenol polyether-10, 0.4 part by weight of citric acid, 0.3 part by weight of sodium hydroxide, and 90.8 parts by weight of purified water.

### Embodiment 2

A povidone iodine solution was prepared by the following steps, and a preparation method was carried out by the following steps:
(1) weighing the following each raw material component by weight:
   10.0 kg of povidone iodine, 0.7 kg of potassium iodide, 0.6 kg of nonylphenol polyether-10, 0.4 kg of citric acid, 0.3 kg of sodium hydroxide, and 88.0 kg of purified water for standby use;
(2) adding povidone iodine into purified water, and performing stirring till complete dissolution, thereby obtaining a first intermediate solution; and
(3) adding a first stabilizer, a second stabilizer, citric acid and sodium hydroxide into the first intermediate solution respectively, and performing stirring, thereby obtaining the povidone iodine solution.

According to the steps, the prepared povidone iodine solution included the following components by weight:
10.0 parts by weight of povidone iodine, 0.7 part by weight of potassium iodide, 0.6 part by weight of nonylphenol polyether-10, 0.4 part by weight of citric acid, 0.3 part by weight of sodium hydroxide, and 88.0 parts by weight of purified water.

### Embodiment 3

A povidone iodine cotton swab was prepared by the following steps:
(1) weighing the following each raw material component by weight:
   15.0 kg of povidone iodine, 0.7 kg of potassium iodide, 0.6 kg of nonylphenol polyether-10, 0.4 kg of citric acid, 0.3 kg of sodium hydroxide, 1.0 kg of glycerin, and 82.0 kg of purified water for standby use;
(2) adding povidone iodine into purified water, and performing stirring till complete dissolution, thereby obtaining a first intermediate solution; and
(3) adding a first stabilizer, a second stabilizer, citric acid, sodium hydroxide and glycerin into the first intermediate solution respectively, and performing stirring, thereby obtaining a povidone iodine solution; and
(4) immersing the cotton swab with the solution by an immersing method according to a proportion of 1-3 ml of the solution absorbed by each cotton swab so as to prepare a povidone iodine cotton swab with enhanced stability.

According to the steps, the prepared povidone iodine cotton swab included:
15.0 parts by weight of povidone iodine, 0.7 parts by weight of potassium iodide, 0.6 parts by weight of nonylphenol polyether-10, 0.4 parts by weight of citric acid, 0.3 parts by weight of sodium hydroxide, 1 part by weight of glycerin, and 82.0 parts by weight of purified water.

### Test and Result

### Experimental Example 1: Stability Test

### 1. Test Method:

Content determination: (1) a proper amount of the povidone iodine solution in the present invention was weighed as a sample to be tested; and (2) a povidone iodine cotton swab sample was taken, the solution in the sample was extruded, and then the extruded solution was tested.

The solution was respectively put into beakers, purified water was added to dilute the solutions to be 80 ml, and the solution was titrated with 0.02 mol/L sodium thiosulfate, wherein sodium thiosulfate per ml was equivalent to 2.538 mg of iodine. The product was put into a constant temperature and humidity chamber with a temperature of 25±2° C. and an RH of 60%±5%, and the iodine content was respectively determined in the 0^{th}, 1^{st}, 3^{rd}, 6^{th}, 9^{th}, 12^{th}, 18^{th}, 24^{th} and 36^{th} months. Specifically, according to the standard test method in the field, the weight of iodine was calculated by consuming the volume of the titration solution, and the concentration of the solution was obtained by dividing the weight of iodine by the weighed weight.

### 2. Test Data Statistics

The effective iodine content of each group of samples was determined in the 0^{th}, 1^{st}, 3^{rd}, 6^{th}, 9^{th}, 12^{th}, 18^{th}, 24^{th} and 36^{th} months. The results are shown in Table 1 below.

TABLE 1
Results of effective iodine content

| Time | Povidone iodine | Povidone iodine |
|---|---|---|
| (Month) | solution | cotton swab |
| 0 | 1.185% | 1.190% |
| 3 | 1.173% | 1.153% |
| 6 | 1.166% | 1.148% |
| 9 | 1.142% | 1.140% |
| 12 | 1.127% | 1.136% |
| 18 | 1.074% | 1.131% |
| 24 | 1.015% | 1.091% |
| 36 | 0.988% | 0.985% |
| RSD % | 6.74 | 5.49 |

### Experimental Example 2: Bactericidal Capacity Test

### 1. Test Method

By referring to ASTME2315, a Time-Kill method was used for evaluating the activity of the povidone iodine solution provided by the present invention as a bactericide, and the antibacterial activity of the povidone iodine solution was detected. The specific steps included:
1.1 A 10⁶ CFU/ml preparation through different strains was prepared.
1.2 0.1 ml of the experimental bacterial suspension was Sucked and placed in 1 ml of a sample.
1.3 The sample was fully reacted with the bacterial solution for 30 s, add a neutralizer and mix uniformly.
1.4 Neutralization was performed for 10 min, then the test solution was diluted, culture was performed in an agar medium, and plate counting was performed.
1.5 One product in each of the above steps was kept for control respectively.
1.6 A PBS buffer solution was used as a blank control group.

The mean of two plates was used as the final result in grouping.

### 2. Test Data Statistics

The results are shown in Table 2 below.

TABLE 2
Bactericidal capacity statistics

| | | | Sample | Control | |
|---|---|---|---|---|---|
| | | Contact | group | group | Eliminating |
| Name of strains | | time | (mean) | (mean) | rate |
| A. baumanii | A. baumanii | 30 s | 6.33E+01 | 4.00E+06 | 99.998% |
| | ATCC 19606 | | | | |
| B. fragilis | B. fragilis | 30 s | <5.00E+00 | 2.90E+08 | >99.999998% |
| | ATCC 43858 | | | | |
| C. albicans | C. albicans | 30 s | 5.00E+03 | 4.65E+06 | 99.89% |
| | ATCC 10231 | | | | |
| C. tropicalis | C. tropicalis | 30 s | <1.17E+01 | 3.50E+06 | 99.9997% |
| | ATCC 750 | | | | |
| E. aerogenes | E. aerogenes | 30 s | <5.00E+00 | 4.05E+06 | >99.99988% |
| | ATCC 29007 | | | | |
| E. faecalis | E. faecalis | 30 s | 6.00E+05 | 1.35E+06 | 55.6% |
| | ATCC 29212 | | | | |
| E. faecium | E. faecium | 30 s | 8.83E+05 | 1.50E+06 | 41.1% |
| | ATCC 51559 | | | | |
| E. coli | E. coli | 30 s | 1.96E+05 | 7.15E+06 | 97.3% |
| | ATCC 11229 | | | | |
| E. coli | E. coli | 30 s | 6.60E+04 | 1.70E+06 | 96.1% |
| | ATCC 25922 | | | | |
| H. influenzae | H. influenzae | 30 s | <1.00E+01 | 1.19E+06 | >99.9992% |
| | ATCC 8149 | | | | |
| K. | K. | 30 s | <8.33E+00 | 1.50E+06 | >99.9994% |
| pneumoniae | pneumoniae | | | | |
| | ATCC 29019 | | | | |
| P. mirabilis | P. mirabilis | 30 s | <5.00E+00 | 5.00E+06 | >99.99990% |
| | ATCC 7002 | | | | |
| P. aeruginosa | P. aeruginosa | 30 s | 1.50E+04 | 6.30E+06 | 99.8% |
| | ATCC 15442 | | | | |
| P. aeruginosa | P. aeruginosa | 30 s | 1.38E+05 | 2.50E+06 | 94.5% |
| | ATCC 27583 | | | | |
| S. marcesens | S. marcesens | 30 s | 1.88E+03 | 3.70E+06 | 99.95% |
| | ATCC 14756 | | | | |
| S. aureus | S. aureus | 30 s | 1.84E+05 | 4.35E+06 | 95.8% |
| | ATCC 6538 | | | | |
| S. aureus | S. aureus | 30 s | 4.67E+05 | 7.00E+06 | 93.3% |
| | ATCC 29213 | | | | |
| S. | S. | 30 s | 9.23E+04 | 2.10E+06 | 95.6% |
| epidermidis | epidermidis | | | | |
| | ATCC 12228 | | | | |
| S. | S. | 30 s | 5.62E+04 | 2.10E+06 | 97.3% |
| haemolyticus | haemolyticus | | | | |
| | ATCC 29970 | | | | |
| S. hominis | S. hominis | 30 s | 9.50E+03 | 3.10E+06 | 99.7% |
| | ATCC 27844 | | | | |
| S. | S. | 30 s | 1.13E+04 | 1.25E+06 | 99.1% |
| saprophyticus | saprophyticus | | | | |
| | ATCC 35552 | | | | |
| S. | S. | 30 s | <2.50E+01 | 1.90E+06 | >99.9987% |
| pneumoniae | pneumoniae | | | | |
| | ATCC 49619 | | | | |
| S. pyogenes | S. pyogenes | 30 s | 1.40E+04 | 7.00E+06 | 99.8% |
| | ATCC 19615 | | | | |

In the above experimental examples, the effective iodine content was taken as an evaluation index, a long-term stability test method was used for determining the effective iodine content, and the stability of the effective iodine content in a povidone iodine-containing solution was determined through relative standard deviation (RSD). The result in table 1 showed that according to the implementation of the present invention, the effective iodine content maintained excellent stability.

In the bactericidal capacity test, the bactericidal capacity of the povidone iodine solution was evaluated through the eliminating rate of 23 bacteria after 30 s contact. Table 2 showed that within 30 s, the povidone iodine solution provided by the present invention had an eliminating rate of more than 99.0% on 14 bacteria, an eliminating rate of more than 90.0% on 21 bacteria, and an eliminating rate of less than 90% on only two bacteria.

The above test result showed that the povidone iodine solution and its product provided by the present invention both have excellent stability and bactericidal capacity.

### Povidone Iodine Gel

In the technical schemes of the present invention, in a first aspect, the invention application provides a water-soluble povidone iodine gel. The povidone iodine gel at least includes povidone iodine, a gel matrix, a stabilizer pack, and a pH regulator pack.

Further, in 100 parts by weight, the povidone iodine gel includes: 5-15 parts by weight of the povidone iodine, 0.3-1.5 parts by weight of the gel matrix, 0.4-1.8 parts by weight of the stabilizer pack, 0.6-1.0 part by weight of the pH regulator pack, and the balance of water, where the povidone iodine gel has a pH value of 1.5-6.5. In embodiments, povidone iodine gel includes: 5-10 parts by weight of the povidone iodine, 0.3-1.0 parts by weight of the gel matrix, 0.4-1.0 parts by weight of the stabilizer pack, 0.6-0.8 part by weight of the pH regulator pack, and the balance of water, where the povidone iodine gel has a pH value of 1.5-4.0. In embodiments, the povidone iodine gel includes: 10-15 parts by weight of the povidone iodine, 1.0-1.5 parts by weight of the gel matrix, 1.0-1.8 parts by weight of the stabilizer pack, 0.8-1.0 part by weight of the pH regulator pack, and the balance of water, where the povidone iodine gel has a pH value of 4.0-6.5.

Further, the povidone iodine raw material has an available iodine content of 9.0-12.0%. In embodiments, the povidone iodine raw material has an available iodine content of 9.0-10.5%. In embodiments, the povidone iodine raw material has an available iodine content of 10.5-12.0%.

Further, the gel matrix is selected from any one of nonionic soluble cellulose ethers or carbomer, preferably hydroxyethyl cellulose, and the hydroxyethyl cellulose has a viscosity of 1,500-65,000 mPa·s. In embodiments, the hydroxyethyl cellulose has a viscosity of 1,500-35,000 mPa·s. In embodiments, the hydroxyethyl cellulose has a viscosity of 35,000-65,000 mPa·s.

Further, the stabilizer pack at least includes a first stabilizer for protecting a polymer and a second stabilizer for reducing decomposition of iodine. Preferably, a nonionic surfactant is selected as the first stabilizer, and potassium iodide or sodium iodide is selected as the second stabilizer. Preferably, the stabilizer pack includes 0.1-1.0 part by weight of the first stabilizer and 0.3-0.8 part by weight of the second stabilizer. In embodiments, the stabilizer pack includes 0.1-0.5 part by weight of the first stabilizer and 0.3-0.5 part by weight of the second stabilizer. In embodiments, the stabilizer pack includes 0.5-1.0 part by weight of the first stabilizer and 0.5-0.8 part by weight of the second stabilizer.

Further, the first stabilizer is selected from any one of nonionic surfactants, preferably nonylphenol polyoxyethylene ether, and more preferably nonylphenol polyoxyethylene ether-9 or nonylphenol polyoxyethylene ether-10.

Further, in the pH regulator pack, an acid may be selected from citric acid and oxalic acid, and an alkali may be selected from sodium hydroxide, potassium hydroxide, and sodium carbonate. Preferably, the pH regulator pack includes 0.3-0.5 part by weight of the citric acid and 0.3-0.5 part by weight of the sodium hydroxide. In embodiments, the pH regulator pack includes 0.3-0.4 part by weight of the citric acid and 0.3-0.4 part by weight of the sodium hydroxide. In embodiments, the pH regulator pack includes 0.4-0.5 part by weight of the citric acid and 0.4-0.5 part by weight of the sodium hydroxide.

Further, the povidone iodine gel further includes glycerol, such as 0.05-1.0 part by weight of glycerol. In embodiments, the povidone iodine gel includes 0.05-0.5 part per weight of glycerol. In embodiments, the povidone iodine gel includes 0.5-1.0 part per weight of glycerol.

In a second aspect, the invention application further provides a method for preparing a water-soluble povidone iodine gel. The method includes the following steps:
(1) providing the following raw materials in 100 parts by weight: 5-15 parts by weight of povidone iodine, 0.3-1.5 parts by weight of a gel matrix, 0.4-1.8 parts by weight of a stabilizer pack, 0.6-1.0 part by weight of a pH regulator pack, and the balance of water, where the stabilizer pack includes a first stabilizer and a second stabilizer, and the pH regulator pack includes an acid and an alkali;
(2) separately dissolving the first stabilizer, the second stabilizer, the acid, and the alkali in a small amount of purified water to obtain a first stabilizer solution, a second stabilizer solution, an acid solution, and an alkali solution;
(3) adding purified water to a liquid preparation tank; slowly adding the gel matrix to the purified water under the condition of homogeneous stirring; conducting vacuumizing to −0.05 MPa to −0.5 MPa; and then conducting stirring to restore to a normal pressure state so as to obtain a first intermediate solution;
(4) adding 5-15 parts by weight of the povidone iodine to the first intermediate solution, and conducting standing; adding the balance of the water, and conducting vacuumizing to 0.05-0.5 MPa; and then homogeneous stirring for mixing and stirring until complete dissolution to restore to a normal pressure state; and
(5) adding the first stabilizer solution and the second stabilizer solution to the liquid preparation tank containing the first intermediate solution, conducting mixing and stirring until complete dissolution, and adding the acid solution and the alkali solution to adjust the pH to 1.5-6.5 so as to obtain the water-soluble povidone iodine gel.

In embodiments, the method include weighing the raw materials.

In a third aspect, the invention application further provides a disinfection preparation containing the above-mentioned povidone iodine gel, where the povidone iodine gel has a content of 5-15 weight %.

Further, the disinfection preparation is concentrated and/or ready-to-use.

In a fourth aspect, the invention application further provides a ready-to-use disinfection cotton swab. A cotton swab is used as a carrier to load a disinfection preparation containing any one of the above-mentioned povidone iodine gel.

In a fifth aspect, the invention application further provides a ready-to-use disinfection paste. A non-woven fabric is used as a carrier to load a disinfection preparation containing any one of the above-mentioned povidone iodine gel.

The povidone iodine gel of the present invention is prepared in specific examples and experimental examples below, and properties of the povidone iodine gel are tested.

### Example 1

A povidone iodine gel was prepared in the following steps:
(1) weighing various raw material components according to the following parts by weight:
weighing 7.5 kg of povidone iodine, 1.0 kg of hydroxyethyl cellulose, 0.5 kg of potassium iodide, 0.5 kg of nonylphenol polyether-10, 0.4 kg of citric acid, 0.3 kg of sodium hydroxide, and 89.8 kg of purified water for later use;
(2) separately dissolving the nonylphenol polyether, the potassium iodide, the citric acid, and the sodium hydroxide in 8.9 kg of the purified water for later use;
(3) adding 40 kg of the purified water to a liquid preparation tank; adding a gel matrix, namely the hydroxyethyl cellulose, to the purified water under the condition of homogeneous stirring at a frequency of 30-60 HZ; conducting vacuumizing to reach an atmospheric pressure of −0.05 Mpa to −0.5 Mpa; and then conducting homogeneous stirring continuously for 10-30 minutes for uniform mixing;
(4) adding 7.5 kg of the povidone iodine to the liquid preparation tank, and conducting standing for more than 1 minute; adding 40 kg of the purified water, and conducting vacuumizing to reach an atmospheric pressure of −0.05 Mpa to −0.5 Mpa; and then conducting homogeneous stirring for mixing and stirring for 40-80 minutes until complete dissolution to remove a vacuum state; and
(5) separately adding a nonylphenol polyether solution, a potassium iodide solution, a citric acid solution, and a sodium hydroxide solution prepared in step (1) to the liquid preparation tank, and conducting mixing and stirring for 5-30 minutes until complete dissolution so as to obtain the povidone iodine gel with a pH value of 1.5.

According to the above steps, the obtained povidone iodine gel includes, in parts by weight:
7.5 parts by weight of povidone iodine, 1.0 part by weight of hydroxyethyl cellulose, 0.5 part by weight of potassium iodide, 0.5 part by weight of nonylphenol polyether-10, 0.4 part by weight of citric acid, 0.3 part by weight of sodium hydroxide, and 89.8 parts by weight of purified water.

### Example 2

Various raw material components were weighed according to the following parts by weight. A povidone iodine gel was prepared in the following steps. A method for preparing the povidone iodine gel includes the following steps:
(1) providing various raw material components according to the following parts by weight:
   weighing 10.0 kg of povidone iodine, 1.0 kg of hydroxyethyl cellulose, 0.5 kg of potassium iodide, 0.5 kg of nonylphenol polyether-10, 0.4 kg of citric acid, 0.3 kg of sodium hydroxide, and 87.3 kg of purified water for later use;
(2) separately dissolving the nonylphenol polyether, the potassium iodide, the citric acid, and the sodium hydroxide in 7.3 kg of the purified water for later use;
(3) adding 40 kg of the purified water to a liquid preparation tank; adding a gel matrix, namely the hydroxyethyl cellulose, to the purified water under homogeneous stirring at a frequency of 30-60 HZ; conducting vacuumizing to reach an atmospheric pressure of −0.05 Mpa to −0.5 Mpa; and then conducting homogeneous stirring continuously for 10-30 minutes for uniform stirring;
(4) adding 10.0 kg of the povidone iodine to the liquid preparation tank, and conducting standing for more than 1 minute; adding 40 kg of the purified water, and conducting vacuumizing to reach an atmospheric pressure of −0.05 Mpa to −0.5 Mpa; conducting homogeneous stirring for mixing and stirring for 40-80 minutes; and after the stirring is completed, removing a vacuum state;
(5) separately adding a nonylphenol polyether solution, a potassium iodide solution, a citric acid solution, and a sodium hydroxide solution prepared in step (1) to the liquid preparation tank, and conducting mixing and stirring for 5-30 minutes so as to obtain a povidone iodine gel solution with a pH value of 6.5; and
(6) injecting the gel solution into a cotton swab to obtain a povidone iodine gel cotton swab with enhanced stability.

In embodiment, the providing step may include weighing the raw materials.

According to the above steps, the cotton swab loaded with the povidone iodine gel is obtained. The povidone iodine gel includes, in parts by weight:
10.0 parts by weight of povidone iodine, 1.0 part by weight of hydroxyethyl cellulose, 0.5 part by weight of potassium iodide, 0.5 part by weight of nonylphenol polyether-10, 0.4 part by weight of citric acid, 0.3 part by weight of sodium hydroxide, and 87.3 parts by weight of purified water.

### Example 3

A povidone iodine gel was prepared in the following steps:
(1) weighing various raw material components according to the following parts by weight:
   weighing 15.0 kg of povidone iodine, 1.0 kg of hydroxyethyl cellulose, 0.5 kg of potassium iodide, 0.5 kg of nonylphenol polyether-10, 0.4 kg of citric acid, 0.3 kg of sodium hydroxide, 1.0 kg of glycerol, and 82.3 kg of purified water for later use;
(2) separately dissolving the nonylphenol polyether, the potassium iodide, the citric acid, and the sodium hydroxide in 7.3 kg of the purified water for later use;
(3) adding 37.5 kg of the purified water to a liquid preparation tank; adding a gel matrix, namely the hydroxyethyl cellulose, to the purified water under homogeneous stirring at a frequency of 30-60 HZ; conducting vacuumizing to reach an atmospheric pressure of −0.05 Mpa to −0.5 Mpa; and then conducting homogeneous stirring continuously for 10-30 minutes until complete dissolution;
(4) adding 15.0 kg of the povidone iodine to the liquid preparation tank, and conducting standing for more than 1 minute; adding 37.5 kg of the purified water, and conducting vacuumizing to reach an atmospheric pressure of 0.05-0.1 Mpa; conducting homogeneous stirring for mixing and stirring for 40-80 minutes; and after the stirring is conducted until complete solution, removing a vacuum state; and
(5) separately adding a nonylphenol polyether solution, a potassium iodide solution, a citric acid solution, a sodium hydroxide solution, and the glycerol prepared in step (1) to the liquid preparation tank, and conducting mixing and stirring for 5-30 minutes so as to obtain a povidone iodine gel solution with a pH value of 4.0.

According to the above steps, the obtained povidone iodine gel includes, in parts by weight:
15.0 parts by weight of povidone iodine, 1.0 part by weight of hydroxyethyl cellulose, 0.5 part by weight of potassium iodide, 0.5 part by weight of nonylphenol polyether-10, 0.4 part by weight of citric acid, 0.3 part by weight of sodium hydroxide, 1 part by weight of glycerol, and 82.3 parts by weight of purified water.

### Results

### Experimental Example 1: Stability Test

### 1. Test Method

Determination of content: An appropriate amount of a product was accurately weighed, put in a beaker, diluted to 80 ml with purified water, and titrated with 0.02 mol/L of sodium thiosulfate, where the sodium thiosulfate per ml is equivalent to 2.538 mg of iodine. The product was put in a constant-temperature and constant-humidity box at 25±2° C. and 60%±5% RH, and the iodine content was determined at 0, 1, 3, 6, 9, 12, 18, 24, and 36 months separately.

### 2. Statistics of Test Data

The available iodine content of samples in each group was determined at 0, 1, 3, 6, 9, 12, 18, 24, and 36 months. Results are as shown in the following Table 1.

TABLE 1
Results of available iodine content

| | Povidone iodine gel |
|---|---|
| Time (month) | solution |
| 0 | 1.193% |
| 3 | 1.176% |
| 6 | 1.155% |
| 9 | 1.133% |
| 12 | 1.112% |
| 18 | 1.069% |
| 24 | 1.034% |
| 36 | 1.003% |
| RSD % | 6.16 |

### Experimental Example 2: Bactericidal Ability Test

### 1. Test Method

With reference to ASTME2315, the activity of the povidone iodine gel provided by the present invention as a bactericide was evaluated by using a Time-Kill method, and the antibacterial activity of the povidone iodine gel was detected. Specific steps are as follows:
1.1, preparing suspensions with a concentration of about 10⁶ CFU/ml from different strains;
1.2, sucking 0.1 ml of an experimental bacterial suspension, and putting the same into 1 ml of a sample;
1.3, allowing the sample to fully react with the bacterial suspension for 30 seconds, and adding a neutralizer for uniform mixing;
1.4, after neutralization is carried out for 10 minutes, diluting a test solution, and conducting culture in an agar culture medium for plate counting;
1.5, preserving a suspension as a control in the above steps; and
1.6, using a PBS buffer as a blank control group.
After grouping, an average of two plates was used as a final result.

### 2. Statistics of Test Data

Results are as shown in the following Table 2.

TABLE 2
Bactericidal capacity statistics

| | | | Sample | Control | |
|---|---|---|---|---|---|
| | | | group | group | |
| | | Contact | (average | (average | |
| Name of strain | | time | value) | value) | Killing rate |
| Acinetobacter | A. baumanii | 30 s | <5.00E+02* | 7.00E+05 | >99.93% |
| baumannii | ATCC 19606 | | | | |
| Bacteroides | B. fragilis | 30 s | <5.00E+00* | 2.28E+07 | >99.9999% |
| fragilis | ATCC 43858 | | | | |
| Candida | C. albicans | 30 s | 4.10E+02 | 1.75E+05 | 99.85% |
| albicans | ATCC 10231 | | | | |
| Candida | C. tropicalis | 30 s | 3.79E+03 | 1.43E+06 | 99.73% |
| tropicalis | ATCC 750 | | | | |
| Enterococcus | E. faecalis | 30 s | 1.10E+06 | 1.98E+06 | 44.30% |
| faecalis | ATCC 29212 | | | | |
| Enterococcus | E. faecium | 30 s | 1.62E+05 | 8.60E+05 | 81.165% |
| faecium | ATCC 51559 | | | | |
| Enterobacter | E. cloacae | 30 s | 1.70E+04 | 7.23E+06 | 99.76% |
| cloacae | ATCC 13047 | | | | |
| Escherichia | E. coli | 30 s | 1.43E+06 | 1.22E+07 | 88.28% |
| coli | ATCC 11229 | | | | |
| Escherichia | E. coli | 30 s | 3.50E+04 | 8.73E+06 | 99.60% |
| coli | ATCC 25922 | | | | |
| Haemophilus | H. influenzae | 30 s | 1.22E+05 | 2.11E+07 | 99.42% |
| influenzae | ATCC 8149 | | | | |
| Klebsiella | K. | 30 s | 2.10E+02 | 1.32E+06 | 99.98% |
| pneumoniae | pneumoniae | | | | |
| | ATCC 29019 | | | | |
| Proteus | P. mirabilis | 30 s | 3.29E+03 | 9.33E+06 | 99.97% |
| mirabilis | ATCC 7002 | | | | |
| Pseudomonas | P. aeruginosa | 30 s | 1.48E+05 | 1.09E+06 | 93.22% |
| aeruginosa | ATCC 15442 | | | | |
| Pseudomonas | P. aeruginosa | 30 s | 9.00E+03 | 1.08E+06 | 99.59% |
| aeruginosa | ATCC 27583 | | | | |
| Serratia | S. marcesens | 30 s | 3.85E+04 | 5.35E+06 | 99.78% |
| marcesens | ATCC 14756 | | | | |
| Staphylococcus | S. aureus | 30 s | 4.05E+04 | 2.75E+05 | 85.27% |
| aureus | ATCC 6538 | | | | |
| Staphylococcus | S. aureus | 30 s | 2.39E+05 | 5.95E+06 | 95.99% |
| aureus | ATCC 29213 | | | | |
| Staphylococcus | S. epidermidis | 30 s | 1.37E+03 | 2.29E+06 | 99.95% |
| epidermidis | ATCC 12228 | | | | |
| Staphylococcus | S. | 30 s | 9.75E+03 | 1.30E+06 | 99.75% |
| haemolyticus | haemolyticus | | | | |
| | ATCC 29970 | | | | |
| Staphylococcus | S. hominis | 30 s | 8.75E+03 | 4.55E+05 | 98.08% |
| hominis | ATCC 27844 | | | | |
| Staphylococcus | S. | 30 s | 2.65E+04 | 2.20E+06 | 98.80% |
| saprophyticus | saprophyticus | | | | |
| | ATCC 35552 | | | | |
| Streptococcus | S. | 30 s | 8.50E+03 | 3.38E+06 | 99.75% |
| pneumoniae | pneumoniae | | | | |
| | ATCC 49619 | | | | |
| Streptococcus | S. pyogenes | 30 s | 2.98E+06 | 5.13E+06 | 41.95% |
| pyogenes | ATCC 19615 | | | | |

*refers to a value lower than a detection limit of a bacterium, and is represented by <.

In the above experimental examples, the effective iodine content was taken as an evaluation index, a long-term stability test method was used for determining the effective iodine content, and the stability of the effective iodine content in a povidone iodine-containing solution was determined through relative standard deviation (RSD). The result in table 1 showed that according to the implementation of the present invention, the effective iodine content maintained excellent stability.

In the bactericidal capacity test, the bactericidal capacity of the povidone iodine solution was evaluated through the eliminating rate of 23 bacteria after 30 s contact. Table 2 showed that within 30 s, the povidone iodine solution provided by the present invention had an eliminating rate of more than 99.0% on 14 bacteria, an eliminating rate of more than 90.0% on 21 bacteria, and an eliminating rate of less than 90% on only two bacteria.

According to the above test results, it can be seen that the povidone iodine gel provided in the invention application has excellent adhesion, stability, and bactericidal ability.

The above descriptions are merely specific examples of the present invention and experimental examples for proving the inventive concept of the invention application, and are not intended to limit the present invention. All transformations which can be directly derived from or associated with the contents disclosed in the present invention by a person of ordinary skill in the art shall be considered to be included in the protection scope of the present invention.

1. A water-soluble povidone iodine gel, wherein the povidone iodine gel at least comprises povidone iodine, a gel matrix, a stabilizer pack, and a pH regulator pack.

2. The povidone iodine gel according to claim 1, wherein in parts by weight, the povidone iodine gel comprises:
5-15 parts by weight of the povidone iodine,
0.3-1.5 parts by weight of the gel matrix,
0.4-1.8 parts by weight of the stabilizer pack,
0.6-1.0 part by weight of the pH regulator pack, and
the balance of purified water;
wherein the povidone iodine gel has a pH value of 1.5-6.5 and a viscosity of 400-5,000 mPa·s.

3. The povidone iodine gel according to claim 1, wherein the gel matrix is selected from any one of nonionic soluble cellulose ethers or carbomer.

4. The povidone iodine gel according to claim 3, wherein the gel matrix is hydroxyethyl cellulose, and the hydroxyethyl cellulose has a viscosity of 1,500-6,500 mPa·s.

5. The povidone iodine gel according to claim 1, wherein the stabilizer pack at least comprises a first stabilizer for protecting a polymer and a second stabilizer for reducing decomposition of iodine, a nonionic surfactant is selected as the first stabilizer, and potassium iodide or sodium iodide is selected as the second stabilizer.

6. The povidone iodine gel according to claim 5, wherein the stabilizer pack comprises 0.1-1.0 part by weight of the first stabilizer and 0.3-0.8 part by weight of the second stabilizer.

7. The povidone iodine gel according to claim 5, wherein the first stabilizer is selected from any one of nonionic surfactants.

8. The povidone iodine gel according to claim 1, wherein the pH regulator pack comprises an acid and an alkali, the acid comprises any one of citric acid and oxalic acid, and the alkali comprises any one of sodium hydroxide, potassium hydroxide, and sodium carbonate.

9. The povidone iodine gel according to claim 1, wherein effective iodine content of the povidone iodine gel decreases by less than 15% after 24 months storage at 25±2° C. and 60%±5% relative humidity.

10. The povidone iodine gel according to claim 1, wherein effective iodine content of the povidone iodine gel decreases by less than 20% after 36 months storage at 25±2° C. and 60%±5% relative humidity.

11. A method for preparing a water-soluble povidone iodine gel, wherein the method comprises the following steps:
(1) providing the following component raw materials according to the following parts by weight: 5-15 parts by weight of povidone iodine, 0.3-1.5 parts by weight of a gel matrix, 0.4-1.8 parts by weight of a stabilizer pack, and 0.6-1.0 part by weight of a pH regulator pack, wherein the stabilizer pack comprises a first stabilizer and a second stabilizer, and the pH regulator pack comprises an acid and an alkali;
(2) separately conducting dissolution in a small amount of purified water to obtain a first stabilizer solution, a second stabilizer solution, an acid solution, and an alkali solution;
(3) adding purified water to a liquid preparation tank; slowly adding the gel matrix to the purified water under the condition of homogeneous stirring; after the addition is completed, conducting vacuumizing to −0.05 MPa to −0.5 MPa; and then conducting stirring continuously to restore to a normal pressure state so as to obtain a first intermediate solution;
(4) adding 5-15 parts by weight of the povidone iodine to the first intermediate solution, and conducting standing; adding the balance of water, and conducting vacuumizing to −0.05 MPa to −0.5 MPa; and then conducting homogeneous stirring for complete mixing and stirring to restore to a normal pressure state; and
(5) adding the first stabilizer solution and the second stabilizer solution to the liquid preparation tank containing the first intermediate solution, conducting mixing and stirring until complete dissolution, and adding the acid solution and the alkali solution to adjust the pH to 1.5-6.5 so as to obtain the water-soluble povidone iodine gel.

12. The method of claim 11, further comprising weighing the component raw materials.

13. A disinfection preparation containing the povidone iodine gel according to claim 1, wherein the povidone iodine gel has a content of 5-15 weight %.

14. The disinfection preparation according to claim 13, wherein the disinfection preparation is concentrated and/or ready-to-use.

15. A ready-to-use disinfection cotton swab, wherein a cotton swab is used as a carrier to load a disinfection preparation containing the povidone iodine gel according to claim 1.

16. A ready-to-use disinfection paste, wherein a non-woven fabric is used as a carrier to load a disinfection preparation containing the povidone iodine gel according to claim 1.

17. A method of killing 80% or more of a bacteria in or on a substrate comprising contacting the substrate with an amount of the povidone iodine gel according to claim 1 in an amount sufficient to kill the bacteria.

18. The method of claim 17, wherein 90% or more of the bacteria are killed.

19. The method of claim 17, wherein 99% or more of the bacteria are killed.

20. The method of claim 17, wherein the bacteria are one or more of the following:
Acinetobacter baumannii
Bacteroides fragilis
Candida albicans
Candida tropicalis
Enterococcus faecium
Enterobacter cloacae
Escherichia coli
Escherichia coli
Haemophilus influenzae
Klebsiella pneumoniae
Proteus mirabilis
Pseudomonas aeruginosa
Pseudomonas aeruginosa
Serratia marcesens
Staphylococcus aureus
Staphylococcus aureus
Staphylococcus epidermidis
Staphylococcus haemolyticus
Staphylococcus hominis
Staphylococcus saprophyticus, and
Streptococcus pneumoniae.

21. A povidone iodine solution, comprising povidone iodine, a stabilizer bag and a pH regulator bag.

22. The povidone iodine solution according to claim 21, wherein based on 100 parts by weight, the povidone iodine solution comprises:
5-15 parts by weight of povidone iodine;
0.4-1.8 parts by weight of the stabilizer bag;
0.6-1.0 part by weight of the pH regulator bag; and the balance water; and
the pH value of the povidone iodine solution is 1.5-6.5.

23. The povidone iodine solution according to claim 21, wherein the stabilizer bag at least comprises a first stabilizer for protecting a polymer and a second stabilizer for reducing iodine decomposition; and a nonionic surfactant is selected as the first stabilizer, and potassium iodide or sodium iodide is selected as the second stabilizer.

24. The povidone iodine solution according to claim 23, wherein the stabilizer bag comprises 0.1-1.0 part by weight of the first stabilizer and 0.3-0.8 part by weight of the second stabilizer.

25. The povidone iodine solution according to claim 23, wherein the first stabilizer is any one selected from the nonionic surfactants.

26. The povidone iodine solution according to claim 21, wherein the pH regulator bag comprises acid and alkali, the acid comprises any one of citric acid and oxalic acid, and the alkali comprises any one of sodium hydroxide, potassium hydroxide and sodium carbonate.

27. The povidone iodine solution according to claim 21, wherein effective iodine content of the povidone iodine gel decreases by less than 10% after 24 months storage at 25±2° C. and 60%±5% relative humidity.

28. The povidone iodine solution according to claim 21, wherein effective iodine content of the povidone iodine gel decreases by less than 20% after 36 months storage at 25±2° C. and 60%±5% relative humidity.

29. A method for preparing a povidone iodine solution, comprising the following steps:
(1) providing the following raw material components by weight:
5-15 parts by weight of povidone iodine; 0.4-1.8 parts by weight of the stabilizer bag; and 0.6-1.0 part by weight of the pH regulator bag;
(2) adding 5-15 parts by weight of povidone iodine into purified water, and performing stirring till complete dissolution, thereby obtaining a first intermediate solution; and
(3) adding the stabilizer bag and the pH regulator bag into the first intermediate solution respectively, and performing stirring, thereby obtaining the povidone iodine solution.

30. The method of claim 29, further comprising weighing the component raw materials.

31. The method according to claim 29, wherein in the method, the stabilizer bag at least comprises the first stabilizer for protecting the polymer and the second stabilizer for reducing iodine decomposition; and a nonionic surfactant is selected as the first stabilizer, and potassium iodide is selected as the second stabilizer.

32. The method according to claim 31, wherein the pH regulator bag comprises acid and alkali, the acid comprises any one of citric acid and oxalic acid, and the alkali comprises any one of sodium hydroxide, potassium hydroxide and sodium carbonate.

33. A disinfection product, taking a non-woven fabric or a medical sponge as a carrier to carry the povidone iodine solution according to claim 21.

34. A method of killing 80% or more of a bacteria in or on a substrate comprising contacting the substrate with an amount of the povidone iodine solution according to claim 21 in an amount sufficient to kill the bacteria.

35. The method of claim 34, wherein 90% or more of the bacteria are killed.

36. The method of claim 34, wherein 99% or more of the bacteria are killed.

37. The method of claim 34, wherein the bacteria are one or more of the following:
A. baumanii
B. fragilis
C. albicans
C. tropicalis
E. aerogenes
E. coli
E. coli
H. influenzae
K. pneumoniae
P. mirabilis
P. aeruginosa
P. aeruginosa
S. marcesens
S. aureus
S. aureus
S. epidermidis
S. haemolyticus
S. hominis
S. saprophyticus
S. pneumoniae, and
S. pyogenes.
